(19) United States
(12) Patent Application Publication
(10) Pub. No.: US 2021/0293785 A1
(43) Pub. Date: Sep. 23, 2021

(54) METHODS FOR PREDICTING STEROID RESPONSIVENESS

(71) Applicant: FLORIDA STATE UNIVERSITY RESEARCH FOUNDATION, INC., TALLAHASSEE, FL (US)

(72) Inventors: AKASH GUNJAN, TALLAHASSEE, FL (US); YUNA SON, TALLAHASSEE, FL (US); ERNEST O. N. PHILLIPS, TALLAHASSEE, FL (US)

(21) Appl. No.: 17/249,903

(22) Filed: Mar. 18, 2021

Related U.S. Application Data
(60) Provisional application No. 62/991,423, filed on Mar. 18, 2020.

(51) Int. Cl. G01N 33/50

(52) U.S. Cl. CPC G01N 33/5008

(57) ABSTRACT

The invention includes a screening method for determining whether or not a subject will respond beneficially to a steroid therapy; a method for selecting a therapy for a subject having a condition, using the screening method; and a method for treating a condition of a subject, using the screening method.

### CROSS-REFERENCE TO RELATED APPLICATION

The present application claims the benefit of U.S. Provisional Application Ser. No. 62/991,423, filed Mar. 18, 2020, which is hereby incorporated by reference herein in its entirety, including any figures, tables, nucleic acid sequences, amino acid sequences, or drawings.

### BACKGROUND OF THE INVENTION

Topical corticosteroids are used extensively to treat a wide range of inflammatory, hyperproliferative and pruritic dermatological disorders such as psoriasis, eczema and dermatitis, while local intra-lesional steroid injections are a mainstay of keloid therapy. However, there is a wide variation in the response of patients to steroid therapy. For example, only about 34% of keloid patients benefit from steroid therapy, while 49% do not respond to it and the remaining 17% of patients actually see a worsening of their keloids upon steroid therapy. This variability in patients' responses to steroids is likely due to individual differences in the patients' genetic or epigenetic makeup, although the genes or epigenetic pathways involved have not yet been identified.

Keloids are characterized by excessive local fibroblast proliferation and overproduction of type I and type III collagen in response to dermal injury.¹⁻³ Keloids cause functional impairment, disfigurement, pain, pruritus, and low quality of life.^{4, 5} Keloids are relatively common in certain racial and ethnic populations with an estimated incidence of ˜1/30 for African Americans and ˜1/625 in the overall US population.⁶ The primary aim of keloid therapy is reduction of fibroblast proliferation and excessive collagen formation. The management of keloids was suboptimal before the use of post-surgical adjunctive therapies with recurrence rates up to 50% with medical management⁷⁻¹¹ and rates up to 100% with surgical treatment alone.¹² Adjunctive preoperative, perioperative, and postoperative therapies including intra-lesional corticosteroids,¹³⁻¹⁶ intra-lesional 5-flourouracil,¹⁷ cryotherapy,¹⁸ and radiation therapy¹⁹⁻²⁵ can reduce the risk of recurrence following surgery by varying degrees. To date there has been no basic research aimed at dissecting the variable effects of steroids in patients receiving them to treat their keloids.

### BRIEF SUMMARY OF THE INVENTION

In order to address the knowledge gap around keloids and responsiveness to treatments for keloids, the inventors analyzed the effects of steroids and various radiation doses and fractionation schemes using X-ray radiation of varying energies on patient-derived keloid explants and fibroblasts. The inventors then tested the optimized radiation parameters from in vitro studies on patients following surgical excision of keloids to determine its efficacy in preventing recurrence. The inventors also compared the efficacy of adjuvant radiation therapy to steroid treatment in blocking keloid fibroblast proliferation and collagen synthesis that drive keloid disease.

Given that steroid therapy often lasts for months and years, the highly variable patient responses to it highlight the dire need for a screening test to determine the patient's response to steroids prior to initiating therapy. To address this need, the inventors have developed a method for screening patient cells that involves culturing them to determine the effects of steroids using an in vitro cell proliferation assay. Cell samples may be obtained by a variety of methods, depending on the target cell type or cell types. For example, cells can be obtained from a small skin biopsy sample (less than 5mm in diameter), or less invasive cell sampling may be used such as obtaining cells via an oral rinse.

An aspect of the invention concerns a method for determining whether or not a subject will respond to a steroid therapy (a screening method), the method comprising: (a) administering a steroid (referred to herein as the “test steroid”), such as triamcinolone acetonide, to a sample of cells obtained from the subject in vitro; (b) determining the effect of the steroid on proliferation of the cells, wherein a decrease in proliferation caused by the steroid is indicative of responsiveness to steroid therapy and steroid therapy may be initiated, wherein no effect on cell proliferation is indicative of a lack of responsiveness to steroid therapy and steroid therapy should be avoided, and wherein an increase in proliferation caused by the steroid is indicative of an adverse effect associated with steroid therapy and steroid therapy should be strictly avoided. In situations in which the results of the screening method indicate that steroid therapy should be avoided, alternative non-steroid therapies may be utilized. For example, in the case of keloid therapy, the inventors have shown that adjuvant radiation therapy is likely to be very effective in preventing keloid recurrence even in steroid-resistant patients. Any subject with a condition in which steroids are a treatment option can benefit from the screening method of the invention.

Another aspect of the invention concerns a method for selecting a therapy for a subject having a condition, comprising: receiving results of an in vitro test for determining the effect of a steroid on proliferation of a sample of cells obtained from the subject, or carrying out the aforementioned screening method; and selecting a steroid therapy for the subject if there is a decrease in cell proliferation caused by the steroid, or withholding steroid therapy from the subject and optionally selecting a non-steroid therapy for the subject if there is no effect on cell proliferation, or an increase in cell proliferation. Any subject with a condition in which steroids are a treatment option can benefit from the treatment selection method of the invention.

Another aspect of the invention concerns a method for treating a condition of a subject, comprising: receiving results of an in vitro test for determining the effect of a steroid on proliferation of a sample of cells obtained from the subject, or carrying out the aforementioned screening method; and administering a steroid therapy to the subject if there is a decrease in cell proliferation caused by the steroid, or withholding steroid therapy from the subject and optionally administering an alternative non-steroid therapy to the subject if there is no effect on cell proliferation, or if there is an increase in cell proliferation. Any subject with a condition in which steroids are a treatment option can benefit from the treatment method of the invention.

As depicted in the flow diagram of FIG. 3, in some embodiments, the method of the invention involves culturing small (˜2 mm) tissue pieces using standard cell culture media and conditions to allow the outgrowth of cells, which takes 1-2 weeks. Depending upon the desired cell type or cell types, non-invasive sampling methods can be utilized, such an oral rinse. The outgrown cells are collected by trypsinization and re-seeded in fresh wells. Upon attachment, the cells are treated in triplicate with or without 10 micromolar Triamcinolone Acetonide (TA), a potent synthetic steroid used widely in the clinic, or varying concentrations of any other test steroid. The viable cells are harvested 4-7 days post-treatment (when the untreated cells start approaching confluency) and counted to determine the effects of the steroid on cell proliferation. The inventors have tested the method of the invention using fibroblast cells derived from 3 normal human skin biopsies as well as 20 surgically excised keloids from patients and exposing them to TA, as well as hydrocortisone, another commonly used steroid in the clinic. The results were remarkably similar to those observed for the response of keloid patients to steroids in the clinic, with 52% of the samples being sensitive (responsive) to the TA treatment, while 39% were non-responsive and 9% were hyperproliferative in TA (which predicts a worsening of condition upon steroid therapy). In summary, our screening test described herein can be used to determine patients' responses to steroids in less than 4 weeks, following which responsive patients can undergo steroid therapy, while others can avoid the potentially harmful steroids altogether and seek other therapies.

No screening tests are currently available to predict a patient's response to topical steroid therapy for dermatological disorders, inhaled steroids for lung disorders, or systemic steroid therapy for autoimmune diseases. Currently, patients with such disorders are offered steroid therapy blindly and based on the empirically determined patient response, the steroid therapy is either continued or discontinued, thereby resulting in a significant potential for harm to the non-responsive patients.

Since the individual differences in patients' responses to steroids are likely to be genetically/epigenetically encoded, as long as viable cells can be obtained from patients, the cells can be tested for their response to steroids. Apart from keloids, the screening method of the invention can be applied more widely to test for responses to all types of steroid therapies using minimally or non-invasive methods for obtaining cells from patients, such as using oral rinses to obtain buccal cells which can then be cultured in vitro for testing, prior to initiation of therapy only if the test predicts a favorable response.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A and 1B. Keloid fibroblasts in culture can be sensitive or resistant to steroid treatment, but are always sensitive to radiation. FIG. 1A shows that keloid fibroblasts from only some patients are sensitive to steroid treatment while others are resistant. Keloid fibroblasts were cultured in the presence or absence of 10 μM Triamcinolone Acetonide (TA) for a week before counting the cells to determine their relative proliferation. Significant sensitivity to TA is indicated by p values shown in black, while significant hyper-proliferation in response to TA is indicated by a p value in red. Interestingly, matched normal skin fibroblasts (HDF-PA) and keloid fibroblasts (PA) obtained from the same individual, or two different keloids obtained from the same patient respond similarly to steroid treatment, suggesting that the different responses of keloids to steroid therapy are likely to reflect the underlying genetic and/or epigenetic differences between patients. (FIG. 1B) The anti-proliferative effects of radiation treatment on keloid fibroblasts are dominant over the effect of steroid treatment. Fibroblasts were treated with or without radiation in the continuous presence or absence of TA as indicated. Cells were counted one week post-irradiation to measure their relative proliferation.

FIGS. 2A-2D. Steroid treatment has a highly variable effect on the outgrowth of cells from keloid explants. Crystal Violet stained plate showing cell outgrowth data for keloid explants derived from patients PC (FIG. 2A), PD (FIG. 2B), and P24R (FIG. 2C) following treatment with or without the steroid TA as described in Methods. The presence of blue dye indicates cell outgrowth from the keloid explant in that well. PC and PD appear to be resistant to TA, while P24R is sensitive to TA. FIG. 2D shows a plot with the quantitation of cell outgrowth data for keloid explants obtained from a total of 4 different patients following TA treatment.

FIG. 3. An embodiment of the screening method of the invention.

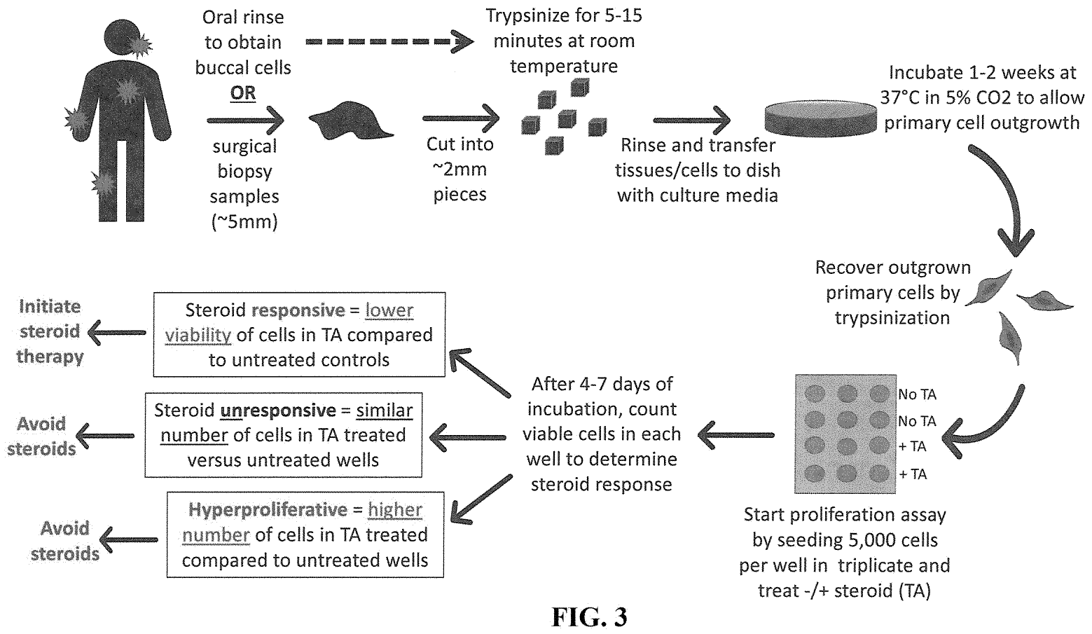

### DETAILED DESCRIPTION OF THE INVENTION

One aspect of the invention concerns a screening method for determining whether or not a subject will respond to a steroid therapy, the method comprising: (a) administering a steroid (referred to herein as the “test steroid”) to a sample of cells obtained from the subject in vitro; (b) determining the effect of the test steroid on proliferation of the cells, wherein a decrease in proliferation caused by the test steroid is indicative of responsiveness to steroid therapy, wherein no effect on cell proliferation is indicative of a lack of responsiveness to steroid therapy, and wherein an increase in proliferation caused by the test steroid is indicative of an adverse effect associated with steroid therapy.

The cells of the cell sample can be from a normal or diseased individual, and can be normal cells or abnormal or diseased cells, e.g., from normal tissue or abnormal or diseased tissue. Without being limited by theory, it is proposed that the response to steroids is genetically/epigenetically encoded and therefore it will be reflected in cells obtained from either normal or diseased tissues.

The cells may be relevant to a condition that the subject has. In some embodiments, the cells are cells of a keloid, such as fibroblasts. Keloid fibroblasts may be from any site in keloid tissue (peri-lesional, intra-lesional and/or extra-lesional sites).

As will be understood by one of skill in the art, there are over 200 cell types in the human body. It is believed that the methods of the subject invention can be used to screen any of these cell types. For example, any cell arising from the ectoderm, mesoderm, or endoderm germ cell layers can be proliferated using methods of the subject invention.

In some embodiments, the sample of cells includes buccal cells, or cells of a surgical biopsy. In some embodiments, the cell sample includes one or more of fibroblasts, epithelial cells, keratinocytes, and melanocytes. Fibroblasts and epithelial cells tend to be easy cells to grow from a tissue where they are found, and as such are very convenient to use in the screening method of the invention.

Advantageously, cells may be sampled and screened using the screening method of the invention at any time. For example, a cell sample from a subject that has a condition that is potentially treatable with a steroid may be screened; however, it also be desirable to screen cell samples from subjects in advance when they are still healthy for their response to steroids, and use this information when the need arises to treat them for a condition that is typically treated using steroids.

Optionally, the screening method further includes, prior to administering the test steroid to the sample of cells, dissociating cells of the sample of cells (e.g., enzymatically, using an enzyme such as trypsin, collagenase, or hyaluronidase; or mechanically, by cutting, pipetting).

Optionally, the method further includes, prior to administering the test steroid to the sample of cells, culturing the cells under conditions sufficient to produce an outgrowth of primary cells.

Optionally, the test steroid is administered to the outgrown primary cells.

Optionally, the determining step of (b) involves conducting a cell proliferation assay.

The subject's cells may be seeded onto a multi-well plate, cell culture dish, or other solid support suitable for accommodating cells, prior to administering the test steroid and determining the effect of the test steroid on cell proliferation. Preferably, the solid support will have an organized array of discrete, separate sample wells in which each sample well will accommodate a sample of cells, and typically be formed of polymer material, glass, or a combination thereof.

The screening method involves administering a test steroid to a sample of cells obtained from the subject in vitro. Without being limited by theory, the inventors propose that the effect of the test steroid on the sample of cells in vitro from a patient will be predictive of the responsiveness of the patient to any steroid therapy with the same mechanism of action (that works through the same cellular pathways) as the test steroid. In some embodiments, the test steroid comprises triamcinolone acetonide or other corticosteroid such as hydrocortisone, prednisone, methylprednisone, dexamethasone, betamethasone, etc. In some embodiments, the test steroid is a mineralocorticoid. In some embodiments, the test steroid is an anabolic steroid.

The test steroid that is administered to cells in vitro may be a single steroid, or may be a combination of two or more steroids that utilize the same or different cellular pathways.

Another aspect of the invention concerns a method for selecting a therapy for a subject having a condition, comprising:

receiving results of an in vitro test for determining the effect of a steroid on proliferation of a sample of cells obtained from the subject, or carrying out, or carrying out the above screening method (i.e., (a) administering a test steroid to a sample of cells obtained from the subject in vitro; (b) determining the effect of the test steroid on proliferation of the cells, wherein a decrease in proliferation caused by the test steroid is indicative of responsiveness to steroid therapy, wherein no effect on cell proliferation is indicative of a lack of responsiveness to steroid therapy, and wherein an increase in proliferation caused by the test steroid is indicative of an adverse effect associated with steroid therapy); and selecting a steroid therapy for the subject if there is a decrease in cell proliferation caused by the test steroid, or withholding steroid therapy from the subject and optionally selecting a non-steroid therapy for the subject if there is no effect on cell proliferation, or an increase in cell proliferation from the test steroid.

Another aspect of the invention concerns a method for treating a condition of a subject, comprising: receiving results of an in vitro test for determining the effect of a steroid on proliferation of a sample of cells obtained from the subject, or carrying out the above screening method (i.e., (a) administering a test steroid to a sample of cells obtained from the subject in vitro; (b) determining the effect of the test steroid on proliferation of the cells, wherein a decrease in proliferation caused by the test steroid is indicative of responsiveness to steroid therapy, wherein no effect on cell proliferation is indicative of a lack of responsiveness to steroid therapy, and wherein an increase in proliferation caused by the test steroid is indicative of an adverse effect associated with steroid therapy; and administering a steroid therapy to the subject if there is a decrease in cell proliferation caused by the test steroid, or withholding steroid therapy from the subject and optionally administering a non-steroid therapy to the subject if there is no effect on cell proliferation, or an increase in cell proliferation from the test steroid.

In the methods of the invention (e.g., method for determining whether or not a subject will respond to a steroid therapy; method for selecting a therapy for a subject having a condition; and method for treating a condition of a subject), the in vitro test for determining the effect of a steroid on the proliferation of the sample of cells obtained from the subject may be carried out by the party potentially administering the steroid therapy to the subject, or the in vitro test may be conducted by a third party, such as a diagnostic laboratory. Results of the in vitro test may be conveyed in any form or format that conveys the results of the test, such as digital or electronic, paper, verbally (in-person or telephone), etc. The in vitro test may be a screening method such as the screening method described herein, i.e., comprising: (a) administering a test steroid to a sample of cells obtained from the subject in vitro; (b) determining the effect of the test steroid on proliferation of the cells, wherein a decrease in proliferation caused by the test steroid is indicative of responsiveness to steroid therapy, wherein no effect on cell proliferation is indicative of a lack of responsiveness to steroid therapy, and wherein an increase in proliferation caused by the test steroid is indicative of an adverse effect associated with steroid therapy.

Steroids are widely used for a variety of medical conditions, in various dosages and delivery routes (Ericson-Neilsen W et al., The Oschner Journal, 2014, 14:203-207; Liu D et al., Allergy, Asthma& Clinical Immunology, 2013, 9(30); Shaikh S et al., ISRN Anesthesiology, 2012, Article ID 985495, which are incorporated herein by reference in their entireties). Any subject with a condition in which steroids are a potential treatment option can benefit from the screening method of the invention. Conditions that may be treated with one or more steroids include, but are not limited to, immune disorders, inflammatory disorders, hyper-proliferative disorders, dermatological disorders, and pain. Examples of immune disorders or inflammatory disorders include rheumatoid arthritis, lupus erythematosus. Examples of dermatological disorders that may be treated using the method of the invention include keloids, psoriasis, eczema, and dermatitis. In some embodiments, the dermatological disorder is a hyper-proliferative skin condition such as one or more keloids, hypertrophic scars, or tumors.

Steroids are used to treat a variety of inflammatory and non-inflammatory conditions. Corticosteroid drugs are chemically modified versions of natural glucocorticosteroids. Examples include cortisone, flurohydrocortisone, hydrocortisone, prednisolone, prednisone, methylprednisolone, dexamethasone, and betamethasone.

Two main classes of corticosteroids include glucocorticoids and mineralocorticoids. Glucocorticoids affect carbohydrate, fat, and protein metabolism, and have anti-inflammatory, immunosuppressive, anti-proliferative, and vasoconstrictive effects. Mineralocorticoids are primarily involved in the regulation of electrolyte and water balance by modulating ion transport.

Corticosteroids are generally grouped into four classes based on their chemical structure. Allergic reactions to one member of a class typically indicate an intolerance to all members of the class (the Coopman classification). Group A corticosteroids (hydrocortisone type) include, for example, hydrocortisone, hydrocortisone acetate, cortisone acetate, tixocortol pivalate, prednisolone, methylprednisolone, and prednisone. Group B corticosteroids (acetonides and related substances) include, for example, amcinonide, budesonide, desonide, fluocinolone acetonide, fluocinonide, halcinonide, and triamcinolone acetonide. Group C corticosteroids (betamethasone type) include, for example, beclomethasone, betamethasone, dexamethasone, fluocortolone, halometasone, and mometasone. Group D corticosteroids include the Group Di corticosteroids (halogenated) such as alclometasone diproprionate, betamethasone dipropionate, betamethasone valerate, clobetasol propionate, clobetasone butyrate, fluprednidene acetate, and mometasone furoate; and Group D₂ (labile prodrug esters), such as ciclesonide, cortisone acetate, hydrocortisone aceponate, hydrocortisone acetate, hydrocortisone buteprate, hydrocortisone butyrate, hydrocortisone valerate, prednicarabate, and tixocortal pivalate.

The steroid to be evaluated as a test steroid and/or active ingredient in a steroid therapy may be a steroid drug. In some embodiments, the steroid therapy may include a glucocorticoid. As used herein, the term “glucocorticoid” refers to a class of steroid hormones that bind to a glucocorticoid receptor, which is present in almost all vertebrate animal cells. The glucocorticoid receptor is also known as a nuclear receptor subfamily 3, group C, member 1 (NR3C1), which is a receptor to which cortisol and other glucocorticoids bind. The glucocorticoid receptor may have an amino acid sequence of NP_000167 (human) and NP_032199 (mouse). The glucocorticoid receptor may be encoded by a nucleotide sequence of NM_000176 (human) and NM_008173 (mouse).

In some embodiments, the steroid drug is one or more selected from among cortisol, hydrocortin, cortisone, prednisolone, methyl prednisolone, triamcinolone, triamcinolone acetonide, paramethasone, dexamethasone, betamethasone, hexoestrol, methimazole, fluocinonide, fluocinolone acetonide, fluorometholone, beclometasone dipropionate, estriol, diflorasone diacetate, diflucortolone valerate, and difluprednate.

Examples of medical conditions that may potentially be treated with corticosteroids includes allergy and respiratory conditions, such as asthma, chronic obstructive pulmonary disease (COPD), allergic rhinitis, atopic dermatitis, hives, angioedema, anaphylaxis, food allergies, drug allergies, nasal polyps, hypersensitivity pneumonitis, sarcoidosis, eosinophilic pneumonia and other pneumonias, and interstitial lung disease. Other conditions include dermatological conditions (e.g., pemphigus vulgaris, contact dermatitis), endocrine conditions (e.g., Addison's disease, adrenal insufficiency, congenital adrenal hyperplasia), gastroenterological conditions (e.g., ulcerative colitis, Crohn's disease, autoimmune hepatitis), hematological conditions (e.g., lymphoma, leukemia, hemolytic anemia, idiopathic thrombocytopenic purpura, multiple myeloma), rheumatological/immunological conditions (e.g., rheumatoid arthritis, systemic lupus erythematosus, polymyalgia rheumatic, polymyositis, dermatomyositis, polyarteritis, vasculitis), ophthalmological conditions (e.g., uveitis, optic neuritis, keratoconjunctivitis), and other conditions, such as multiple sclerosis relapses, organ transplant rejection, nephrotic syndrome, chronic hepatitis (flare ups), cerebral edema, IgG4-related diseases, prostate cancer, tendinosis, and lichen planus.

In some embodiments, the condition is an autoimmune disorder, such as rheumatoid arthritis, lupus erythematosus, or multiple sclerosis. In some embodiments, the condition is an inflammatory disorder such as atopic dermatitis, chronic obstructive pulmonary disease, or asthma.

The condition may be of any severity (e.g., mild, moderate, or severe), and may be acute or chronic.

In some embodiments, the condition is selected from among nephrotic syndrome, myasthenia gravis, lupus nephritis, cerebritis, dermatopolymyositis, temporal arteritis, immune hemolytic anemia, sarcoidosis, and systemic vasculitides.

The condition may be a cancer. The cancer may be a solid tumor such as breast cancer or prostate cancer, or a hematologic malignancy. In some embodiments, the cancer is acute lymphocytic leukemia, Hodgkin's lymphoma, non-Hodgkin's lymphoma, myeloma, or chronic lymphocytic leukemia.

In some embodiments, the subject has a dermatologic condition and the steroid therapy is topical steroid therapy. In some embodiments, the subject has a respiratory or lung disorder, and the steroid therapy is an inhaled steroid therapy. In some embodiments, the subject has an autoimmune disease, and the steroid therapy is a systemic steroid therapy.

The alternative non-steroidal therapy may be any non-steroid agent (i.e., non-steroid and not a steroid derivative) or non-steroidal treatment, such as a non-steroid chemical compound, radiation therapy, cryotherapy, surgery, or a combination of two or more of the foregoing. In some embodiments, the non-steroid alternative is a non-steroid anti-inflammatory agent, or non-steroid immune-suppressive agent, or non-steroid immunomodulator. Examples of conditions and corresponding steroid therapies and some alternative non-steroid therapies are shown in the table below.

TABLE 1

| Condition | Steroid Therapies | Non-Steroid Alternatives |
|---|---|---|
| Rheumatoid | Prednisone, | methotrexate, sulfasalazine, |
| arthritis | hydrocortisone, | leflunomide, (Hydroxy-) |
| | prednisolone, | chloroquine, tofacitinib, |
| | dexamethasone, | baricitinib, etanercept, |
| | methylprednisolone, | infliximab, adalimumab, |
| | triamcinolone, | golimumab, certolizumab, |
| | betamethasone | tocilizumab, sarilumab, |
| | | abatacept, rituximab |
| Lupus | Prednisone, prednisolone, | belimumab, tabalumab, |
| erythematosus | methylprednisolone, | blisibimod, atacicept, |
| | cortisone, | abetimus sodium, rituximab, |
| | hydrocortisone, | ocrelizumab, ofatumumab, |
| | | obinutuzumab, epratuzumab, |
| | | rontalizumab, sifalimumab |
| | | Anifrolumab, tocilizumab, |
| | | sirukumab, eculizumab, |
| | | ruplizumab/ toralizumab, |
| | | dapirolizumab, abatacept |
| Severe atopic | Clobetasol, betamethasone, | topical calcineurin inhibitor, |
| dermatitis | mometasone, fluticasone, | topical PDE4 inhibitor, |
| | methylprednisolone, | tofacitinib, dupilumab, |
| | hydrocortisone, | cyclosporine, phototherapy, |
| | flumethasone | methotrexate, mycophenolate |
| | | mofetil (MMF), azathioprine, |
| | | upadacitinib, PF-04965842, |
| | | baricitinib, nemolizumab, |
| | | lebrikizumab, tralokinumab, |
| | | fezakinumab, ustekinumab |
| Multiple | Methylprednisolone, | topical tocoretinate, |
| sclerosis | prednisone, | copaxone natalizumab, |
| | dexamethasone | alemtuzumab, daclizumab, |
| | | mitoxantrone teriflunomide, |
| | | delayed-release dimethyl |
| | | fumarate (DMF), |
| | | fingolimod, rituximab, |
| | | ocrelizumab, ofatumumab, |
| | | laquinimod, cladribine, |
| | | siponimod, ozanimod, |
| | | transplantation of autologous |
| | | bone marrow, opicinumab, |
| | | MSC engraftment, |
| | | autologous MSCs |
| Asthma | prednisone | albuterol, levalbuterol, |
| | | terbutaline, metaproterenol |
| | | pirbuterol, salmeterol, |
| | | formoterol, zafirlukast, |
| | | montelukast, zileuton |
| | | ipratropium, tiotropium, |
| | | aclidinium, umeclidinium |
| | | glycopyrronium, |
| | | omalizumab, mepolizumab, |
| | | reslizumab benralizumab, |
| | | dupilumab, tezepelumab |

The test steroid may be the same steroid that serves as the active agent in the potential steroid therapy under consideration for a subject, or the test steroid may be different but of the same class as the steroid that serves as the active agent in the potential steroid therapy under consideration, or the test steroid may be of a different class of steroid than the steroid that serves as the active agent in the potential steroid therapy under consideration. As indicative above, without being limited by theory, the inventors propose that the effect of the test steroid on the sample of cells in vitro from a patient will be predictive of the responsiveness of the patient to any steroid therapy with the same mechanism of action (that works through the same cellular pathways) as the test steroid. Therefore, for example, the effect of TA or other corticosteroid on the patient's cells in vitro will be predictive of the response to the test corticosteroid or to any other corticosteroid, on that patient. In some embodiments, the test steroid is a mineralocorticoid. In some embodiments, the test steroid is an anabolic steroid.

In some embodiments, the test steroid is a corticosteroid and the potential steroid therapy comprises a corticosteroid. In some embodiments, the test steroid is a mineralocorticoid and the potential steroid therapy comprises a mineralocorticoid. In some embodiments, the test steroid is an anabolic steroid and the potential steroid therapy comprises an anabolic steroid.

The test steroid that is administered to cells in vitro may be a single steroid, or may be a combination of two or more steroids that utilize the same or different cellular pathways, and the steroid therapy may include a single steroid as the active agent or a combination of two or more steroids that utilize the same or different cellular pathways.

Techniques for formulation and administration of drugs, including steroids and non-steroids may be found in the latest edition of “Remington's Pharmaceutical Sciences,” Mack Publishing Co., Easton, Pa., which is herein fully incorporated by reference.

The steroid therapy may include administration of a steroid prodrug.

Suitable routes of administration for steroids and the pharmaceutical compositions containing them may, for example, include topical, local injection, oral, inhalation, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections.

As used herein, the terms “a” and “an” preceding an element or component of the invention are intended to be nonrestrictive regarding the number of instances, i.e., occurrences of the element or component. Therefore, “a” or “an” should be read to include one or at least one, and the singular word form of the element or component also includes the plural unless the number is obviously meant to be singular. For example, “a steroid” or “a test steroid” is inclusive of an individual steroid and a combination of two or more steroids.

As used herein, the term “active ingredient” refers to the agent accountable for the intended biological effect (e.g., a steroid agent, or non-steroid agent). As used herein, the phrases “physiologically acceptable carrier” and “pharmaceutically acceptable carrier,” which may be used interchangeably, refer to a carrier or a diluent that does not cause significant irritation to the subject and does not abrogate the biological activity and properties of the administered agent.

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. The term “administration” is inclusive of a self-administration and administration by another individual, such as by personnel of a health care provider. Administration can be local at a particular anatomical site, such as a site of infection or flare up, or systemic. Administration of steroid therapy and non-steroid therapy can be continuous or at distinct levels as can be readily determined by a person skilled in the art. In the context of administering a test steroid to a cell or sample of cells in vitro, administering means bringing the test steroid and cell(s) into contact with one another by any method.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “contacting” in the context of contacting a cell of a sample with at least one compound such as a test steroid in vitro or in vivo means bringing steroid into contact with the cell, or vice-versa, or any other manner of causing the steroid and the cell to come into contact.

As used herein, the term “excipient” refers to an inert substance added to a pharmaceutical composition to further facilitate administration of an active ingredient. Examples of excipients include, but are not limited to, calcium carbonate, calcium phosphate, various sugars and types of starch, cellulose derivatives, gelatin, vegetable oils, and polyethylene glycols.

As used herein, the terms “subject”, “patient”, and “individual” refer to a human or non-human animal. Typically, the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. The subject may be any age or gender.

As used herein, the term “steroid” includes steroids and steroid derivatives. In some embodiments, the steroid is a corticosteroid. In some embodiments, the steroid is a prodrug. The steroid may be a singular steroid or a combination of two or more steroids that utilize the same or different cellular pathways. In some embodiments, the steroid is a biologically active organic compound having a core structure composed of four rings. In some embodiments, the steroid has a typical steroid core structure composed of seventeen carbon atoms, bonded in four fused rings: three six-member cyclohexane rings and one five-member cyclopentane ring. This structure encompasses many steroids that vary by the functional groups attached to this four-ring core and by the oxidation state of the rings.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset, or development, or recurrence, or progression, of the disease or disorder). Thus, in some embodiments, the subject has the condition at the time the steroid therapy or non-steroid therapy is administered, such that the therapy would be administered to treat an existing condition. In other embodiments, the subject does not have the condition at the time the steroid therapy or non-steroid therapy is administered, such that the therapy would be administered to prevent or delay onset or recurrence of the condition (prophylaxis).

### MATERIALS AND METHODS

Culture of keloid derived primary fibroblast cells. Surgically excised keloid and normal skin explants were obtained from patients under Florida State University IRB (2016.19175 and 2017.22173). The explants were cut into small pieces, digested with trypsin, washed with and cultured in DMEM containing 10% fetal bovine serum and Antibiotic/Antimycotic solution maintained at 37° C. and 5% CO2. Outgrowth of only primary dermal fibroblasts occurred under our culture conditions and these were recovered by trypsinization for use in experiments at low passage. The keloid fibroblasts are morphologically identical to commercially obtained normal primary Human Dermal Fibroblasts (HDF) (Cell Applications, Cat.#106-05a) used in our controls.

Cell proliferation assays. To determine relative cell survival following radiation or steroid treatments, 7,500 fibroblasts were seeded per well in triplicate in 12-well plates. Following attachment, the cells were treated with steroids or irradiated using a X-Rad 320 irradiator. Surviving fibroblasts were counted on a Coulter counter 7 days post-treatment.

Keloid explant outgrowth. To determine if steroids or radiation could block the outgrowth of cells from keloid explants, fresh keloid tissue roughly 4 mm³ in size was placed skin side up in culture media in multi-well plates prior to irradiation or steroid treatment. The tissue pieces were cultured for 4 weeks and then removed prior to fixing the outgrown cells and staining with 0.5% w/v Crystal Violet.

Post-surgical radiation treatment of patients. Post-surgical irradiation on patients was performed using a Sensus SRT-100 irradiator with a 0 mm margin to avoid any unnecessary radiation exposure, with the area surrounding the wound protected by 0.763 mm think lead shield held in place using skin tape. All patients were observed by two dermatologists and there was complete concurrence between their evaluations. Pre-operative, post-operative and follow-up photographs were taken.

Statistical analysis. Significant differences between groups was analyzed by one-way ANOVA, or Student's t-test with repeated measures using Excel and R software. A Tukey's post hoc analysis was used in ANOVA analysis of significant effects. The difference between the means for all conditions is significant when p≤0.05.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### Radiation Blocks the Proliferation of Steroid Resistant Keloid Fibroblasts

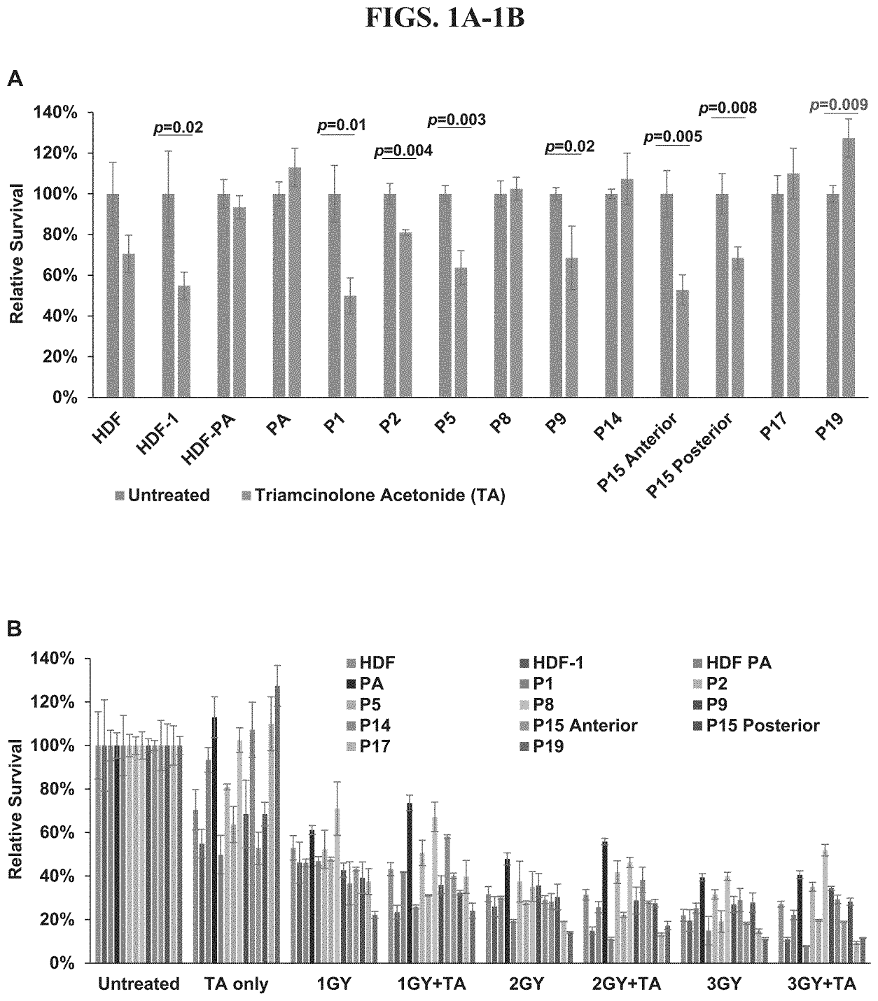

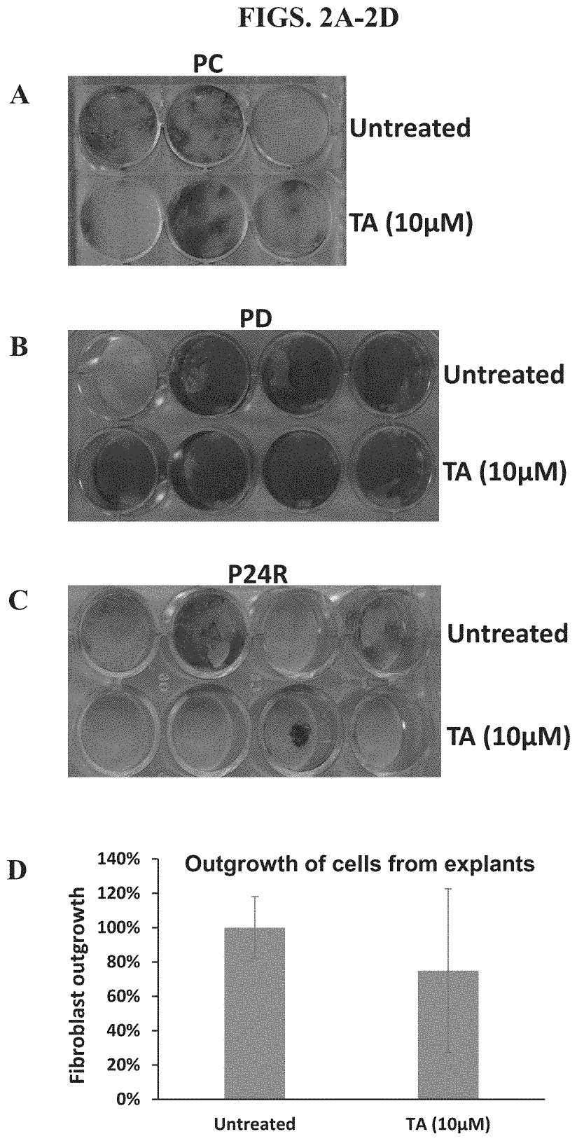

Steroids have been used empirically as a primary or adjuvant therapy for keloids with highly variable results^{13-16, 29}. To better understand this variability, we studied the effects of a potent synthetic glucocorticoid Triamcinolone Acetonide (TA) on keloid explants (FIGS. 2A-2D) and fibroblasts (FIG. 1A). TA treatment resulted in either a modest reduction in proliferation (50% of samples, steroid sensitive), no significant effects on proliferation (40%, steroid insensitive), or hyperproliferation of some samples (10%). Remarkably, this in vitro data is nearly identical to the actual response to steroids observed in the clinic²⁹ and in an online survey³⁰ of over 800 patients. Since steroids and radiation are often used in combination in the clinic for keloid management, we also tested their combined effects on keloid fibroblasts.

Radiation and steroid treatment had synergistic anti-proliferative effects on steroid sensitive keloid fibroblasts (FIG. 1B). Remarkably, radiation not only inhibited proliferation of steroid insensitive keloid fibroblasts, but the anti-proliferative effects of radiation were dominant over the effects of steroids on keloid fibroblasts that exhibit hyperproliferation in response to steroids.

### Example 2

### A Single Dose of 8 Gy, 50 kV X-Rays is Effective In Blocking Keloid Recurrence In Patients Following Surgical Excision

A simple regression analysis of the data suggested that a single dose of 8 Gy, 50 kV X-rays maybe effective for adjuvant keloid therapy following surgical excision to prevent recurrence. The inventors tested this following the excision of 20 keloids from various locations in 15 African-American patients (Table 3). Radiation was delivered at an average of 34 days (range 6-103 days) post-surgery, once the wound had sufficiently granulated (as judged by the beefy-red tissue over the wound bed) Other studies emphasize radiation delivery immediately or within 72 hours of keloid surgery,^{23, 31} which could delay wound healing. Significant adverse effects such as fibrosis or desquamation are not expected with low dose of superficial radiation.

Mild hyperpigmentation at the site of radiation was observed in all patients which resolved without any intervention over time. 75% of the excised keloids were treated for pruritus following radiation with one or more injections of 0.2-1 cc of Kenalog 10 or 20 (Table 3). Two keloids that had received intralesional steroid injections without any benefit are still free of recurrence 22 months after radiation. There was no recurrence for 12 keloids in 9 patients at the 6 months follow up visit, while 5 patients with 7 keloids could not be contacted. One patient presented again with a 5 mm keloid at the distal end of the excision site 4-months later, suggesting that this could either be a recurrence due to a failure of our 0 mm radiation margin, or a de novo keloid. Hence, at 6 months, of the keloids that we could follow up, at worse the keloid recurrence rate was 1 in 13, or 7.69%, which is much lower compared to the rates reported for adjuvant therapies following keloid excision. Only 8 patients with 10 keloids could be contacted for follow up beyond 6 months and none of them had a recurrence after a mean follow up of 22 months (range 10-42 months) (Table 3).

No standardized treatment schedule exists for the use of post-surgical radiation despite the use of radiation in keloid therapy for decades.26, 32, 33 Herein, the inventors have provided the first systematic analysis of the effect of different radiation parameters and steroids on keloid fibroblast proliferation in vitro, and then used this data to develop and test an evidence-based radiation treatment schedule to prevent post-excision keloid recurrence.

The data derived from the analysis of keloid explants and/or fibroblasts obtained from 18 patients were very similar in their response to radiation irrespective of original keloid location or race, age and sex of the patient (Table 2). This fairly uniform response is unlikely to be due to the similar adaptations the cells may undergo during culture. This view is supported by the high variability in their response to steroids (FIG. 1A) and the predominant pathways mediating cell death in keloid fibroblasts from different patients. Overall, the data suggest that the primary keloid fibroblasts in culture retain the patient-specific genetic and epigenetic features that determine responses to therapy, and similar to prior studies³⁴⁻⁴⁰, are an appropriate model for the study of keloid biology and the evaluation of keloid therapeutics in vitro.

TABLE 2
Patient derived normal and keloid fibroblasts used in this study.

| Patient | Race | Location | Sex | Age | Comments |
|---|---|---|---|---|---|
| P1 | African American | Ear | F | 55 | Keloid |
| P2 | African American | Chest | F | 69 | Keloid |
| P5 | Hispanic | Scalp | F | 23 | Keloid |
| P6 | African American | Ear | M | 27 | Keloid |
| P8 | African American | Ear | F | 28 | Keloid |
| P9 | African American | Ear | M | 21 | Keloid |
| P12 | African American | Ear | M | 28 | Keloid |
| P14 | African American | Chest | F | 51 | Keloid |
| P15 | African American | Ear | M | 23 | Keloid |
| P16 | African American | Ear | F | 37 | Keloid |
| P17 | Caucasian | Ear | F | 23 | Keloid |
| P19 | African American | Ear | F | 17 | Keloid |
| P22 | African American | Face | M | 40 | Keloid |
| P23 | African American | Ear | M | 57 | Keloid |
| P24L & P24R | African American | Neck | M | 51 | Keloid |
| P25 | African American | Face | M | 40 | Keloid |
| PA | African American | Ear | F | 21 | Keloid |
| HDFPA | African American | Ear | F | 21 | Normal skin |
| PC | African American | Ear | M | 38 | Keloid |
| HDF1 | Caucasian | Ear | F | 48 | Normal skin |
| HDF | Caucasian | Face | F | 50 | Normal Skin |

Type I collagen mRNA levels in keloid fibroblasts have been reported to drop after exposure to 15 Gy of X-ray radiation⁴¹. However, in a different study, radiation did not alter collagen protein levels in mouse fibroblasts.⁴²

The results herein on the variable effects of steroids on keloid fibroblasts and explants (FIGS. 1A and 1B; FIGS. 2A-2D) suggest that steroids should be used with caution in keloid therapy, perhaps only after ascertaining sensitivity of the keloid cells to the steroid. Our data show that it is possible to test cells from patients for their response to steroids in vitro, following which treatment could be initiated if the cells are sensitive. These data also reinforce the suitability of using patient derived keloid dermal fibroblasts in vitro to study keloid biology and test the effects of different therapies as the cultured cells seem to recapitulate the responses of keloids to treatments in patients^{29, 30} .

The high radiation doses (15-20 Gy are common, while doses ≥30 Gy are still used), coupled with higher radiation energies and multiple fractions currently used in keloid therapy greatly increase the potential risk of adverse effects of radiation.³² Our in vitro data on radiation sensitivity of keloid fibroblasts, including those that are steroid resistant, highlight the need to evaluate the therapeutic effects of lower doses of radiation for adjuvant keloid therapy. Remarkably, treatment of 15 patients with 20 keloids with a single 8 Gy, 50 kV dose of radiation resulted in a very low recurrence rate of 7.69% at six months and no additional recurrences among the 8 patients with 10 keloids that we able to follow for an average of 22 months (Table 3). Importantly, radiation delivered at any time up to 3 months post-surgery (i.e., after the surgical wounds have healed), is effective in preventing recurrence and doing so circumvents any effect of radiation on wound healing. Although some of the patients that we were unable to follow up with may have had a recurrence, we suspect that it is more likely that they are still free of keloids and hence do not feel the need to be revaluated. Hence, we conclude that post-surgical adjuvant keloid therapy with a single low dose of superficial X-ray radiation delivered post-wound healing will greatly reduce recurrence of both steroid sensitive and resistant keloids, while dramatically reducing potential adverse effects associated with higher doses of radiation.

Additionally, radiation delivery in a single fraction will improve patient compliance and decrease costs as only one office visit would be required.

TABLE 3
Patients treated with a single dose of 8 Gy, 50 kV X-ray radiation following surgical excision of their keloids.

| | | | | | | | Time | | Intralesional | | Time |
|---|---|---|---|---|---|---|---|---|---|---|---|
| | | | | | Intralesional | | between | | TA | | without |
| | | | | Keloid | triamcinolone | | excision | | following | Recurrence | recurrence |
| | | | | size (in | acetonide | | and | Recurrence | radiation | at 1 year | (in months |
| | Keloid | | | mm) at | (TA) prior to | Date of | radiation | at 6-month | to relieve | (assessed via | as of |
| Patient | location | Sex | Age | excision | excision | excision | (days) | follow-up | itching | phone call) | Jan. 30, 2020) |
| 1 | R upper | F | 65 | 20 × 20 | No | Aug. 17, 2016 | 28 | No | Monthly × 3 | No | 42 |
| | back | | | | | | | | | recurrence | |
| 2a | R neck | M | 50 | 40 | No | Jun. 28, 2018 | 25 | No | Once | Unable to | Unknown |
| | | | | | | | | | | reach. Last | |
| | | | | | | | | | | follow up on | |
| | | | | | | | | | | Nov. 6, 2018. | |
| 2b | L neck | M | 50 | 33 | No | Jun. 28, 2018 | 25 | No | Once | Unable to | Unknown |
| | | | | | | | | | | reach. Last | |
| | | | | | | | | | | follow up on | |
| | | | | | | | | | | Nov. 6, 2018. | |
| 3a | Occipital | M | 39 | 54 × 74 | No | Mar. 29, 2018 | 55 | No | Once | No | 22 |
| | scalp | | | | | | | | | recurrence. | |
| 3b | L cheek | M | 39 | 34 × 24 | No | Mar. 29, 2018 | 55 | No | Once | No | 22 |
| | | | | | | | | | | recurrence. | |
| 4 | Chest | M | 48 | 35 | No | Mar. 22, 2018 | 13 | No | Monthly × 2 | No | 22 |
| | | | | | | | | | | recurrence. | |
| 5 | Abdomen | F | 50 | 25 | No | Mar. 22, 2018 | 7 | Unknown | No | Unable to | Unknown |
| | | | | | | | | | | reach. Last | |
| | | | | | | | | | | follow up on | |
| | | | | | | | | | | May 11, 2018. | |
| 6 | R ear | F | 52 | 16 | Yes; | Mar. 14, 2018 | 8 | No | Monthly × 2 | No | 22 |
| | | | | | Nov. 20, 2017 | | | | | recurrence. | |
| 7 | L earlobe | M | 55 | 60 × 30 | No | Mar. 29, 2018 | 6 | Unknown | Monthly × 3 | Unable to | Unknown |
| | | | | | | | | | | reach. Last | |
| | | | | | | | | | | follow up on | |
| | | | | | | | | | | Jul. 17, 2018. | |
| 8a | R cheek | M | 41 | 70 × 20 | No | Mar. 14, 2019 | 7 | No | Monthly × 9 | No recurrence | 10 |
| | | | | | | | | | | (at 10 months) | |
| 8b | L cheek | M | 41 | 84 | No | Feb. 14, 2019 | 13 | No | Monthly × 9 | No recurrence | 11 |
| | | | | | | | | | | (at 11 months) | |
| 9 | R jawline | F | 24 | 44 | No | Mar. 20, 2019 | 8 | No | Monthly × 4 | No recurrence | 10 |
| | | | | | | | | | | (at 10 months) | |
| 10 | R breast | F | 52 | 90 | No | Nov. 14, 2018 | 103 | No | No | No | 14 |
| | | | | | | | | | | recurrence. | |
| 11 | Posterior | M | 35 | 135 | Yes; | Mar. 29, 2018 | 61 | No | No | No | 22 |
| | scalp | | | | Mar. 15, 2018 | | | | | recurrence. | |
| 12 | R occipital | M | 24 | 90 | No | May 31, 2018 | 41 | Yes | No | Keloid | Not |
| | scalp | | | | | | | | | returned | applicable |
| | | | | | | | | | | September 2018 | |
| | | | | | | | | | | and re-excised | |
| | | | | | | | | | | February 2019. | |
| 13a | R chest | F | 45 | 63 | No | Aug. 8, 2018 | 82 | Unknown | Once | Unable to | Unknown |
| | | | | | | | | | | reach. Last | |
| | | | | | | | | | | follow up on | |
| | | | | | | | | | | Feb. 13, 2019. | |
| 13b | L posterior | F | 45 | 42 | No | Aug. 8, 2018 | 82 | Unknown | Once | Unable to | Unknown |
| | shoulder | | | | | | | | | reach. Last | |
| | | | | | | | | | | follow up on | |
| | | | | | | | | | | Feb. 13, 2019. | |
| 13c | L chest | F | 45 | 120 | No | Nov. 28, 2018 | 48 | Unknown | Once | Unable to | Unknown |
| | | | | | | | | | | reach. Last | |
| | | | | | | | | | | follow up on | |
| | | | | | | | | | | Feb. 13, 2019. | |
| 14 | R pubis | F | 56 | 35 | No | Jan. 25, 2018 | 8 | Unknown | No | Unable to | Unknown |
| | | | | | | | | | | reach. No | |
| | | | | | | | | | | follow up. | |
| 15 | R ear | M | 22 | 10 | No | Dec. 28, 2017 | 6 | Unknown | Once | Unable to | Unknown |
| | | | | | | | | | | reach. Last | |
| | | | | | | | | | | follow up on | |
| | | | | | | | | | | Mar. 13, 2018. | |

### Exemplified Embodiments

Examples of embodiments of the invention include, but are not limited to:

Embodiment 1. A method for determining whether or not a subject will respond to a steroid therapy, the method comprising: (a) administering a steroid to a sample of cells obtained from the subject in vitro; (b) determining the effect of the steroid on proliferation of the cells, wherein a decrease in proliferation caused by the steroid is indicative of responsiveness to steroid therapy, wherein no effect on cell proliferation is indicative of a lack of responsiveness to steroid therapy, and wherein an increase in proliferation caused by the steroid is indicative of an adverse effect associated with steroid therapy.

Embodiment 2. The method of embodiment 1, wherein the sample of cells comprise one or more cell types selected from among fibroblasts, epithelial cells, keratinocytes, and melanocytes.

Embodiment 3. The method of embodiment 1, wherein the subject has a condition, for which a steroid is a treatment option, at the time the sample of cells is obtained from the subject.

Embodiment 4. The method of embodiment 1, wherein the subject does not have a condition, for which a steroid is a treatment option, at the time the sample of cells is obtained from the subject.

Embodiment 5. The method of embodiment 1, wherein cells of the cell sample comprise normal cells.

Embodiment 6. The method of embodiment 1, wherein the cells of the cell sample comprise abnormal or diseased cells.

Embodiment 7. The method of embodiment 1, wherein the sample of cells comprises cells of a keloid. Embodiment 8. The method of embodiment 1, wherein the sample of cells comprises buccal cells, or cells of a surgical biopsy.

Embodiment 9. The method of embodiment 1, further comprising, prior to administering the steroid to the sample of cells, dissociating cells of the sample of cells (e.g., enzymatically, using an enzyme such as trypsin, collagenase, or hyaluronidase; or mechanically, by cutting, pipetting).

Embodiment 10. The method of any preceding embodiment, further comprising, prior to administering the steroid to the sample of cells, culturing the cells under conditions sufficient to produce an outgrowth of primary cells.

Embodiment 11. The method of embodiment 10, wherein said administering of (a) comprises administering the steroid to the outgrown primary cells.

Embodiment 12. The method of any preceding embodiment, wherein said determining of (b) comprises conducting a cell proliferation assay.

Embodiment 13. The method of any preceding embodiment, wherein the cells are seeded onto a solid support (e.g., a multi-well plate or cell culture dish) prior to administering the steroid and determining the effect of the steroid on cell proliferation.

Embodiment 14. The method of any preceding embodiment, wherein the steroid comprises a corticosteroid (e.g., triamcinolone acetonide, hydrocortisone, prednisone, methylprednisone, dexamethasone, or betamethasone).

Embodiment 15. A method for selecting a therapy for a subject having a condition, comprising:

carrying out the method of any one of embodiments 1 to 14, or receiving results of an in vitro test for determining the effect of a steroid on proliferation of a sample of cells obtained from the subject; and

selecting a steroid therapy for the subject if there is a decrease in cell proliferation caused by the steroid, or withholding steroid therapy from the subject and optionally selecting a non-steroid therapy for the subject if there is no effect on cell proliferation, or an increase in cell proliferation.

Embodiment 16. A method for treating a condition of a subject, comprising:

carrying out the method of any one of embodiments 1 to 15, or receiving results of an in vitro test for determining the effect of a steroid on proliferation of a sample of cells obtained from the subject; and

administering a steroid therapy to the subject if there is a decrease in cell proliferation caused by the steroid, or withholding steroid therapy from the subject and optionally administering a non-steroid therapy to the subject if there is no effect on cell proliferation, or an increase in cell proliferation.

Embodiment 17. The method of any preceding embodiment, wherein the subject has an immune disorder, inflammatory disorder, hyper-proliferative disorder, or dermatological disorder.

Embodiment 18. The method of any preceding embodiment, wherein the subject has a keloid.

Embodiment 19. The method of any preceding embodiment, wherein the steroid therapy comprises topical steroid therapy.

Embodiment 20. The method of any one of embodiments 1 to 19, wherein the steroid therapy comprises inhalant steroid therapy.

Embodiment 21. The method of any one of embodiments 1 to 19, wherein the steroid therapy comprises systemic steroid therapy.

Embodiment 22. The method of any preceding embodiment, wherein the steroid therapy comprises corticosteroid therapy.

Embodiment 23. The method of any preceding embodiment, wherein the subject has the condition at the time the sample of cells is obtained from the subject.

Embodiment 24. The method of any one of embodiments 1-22, wherein the subject does not have the condition at the time the sample of cells are obtained from the subject.

Embodiment 25. The method of any one of embodiments 1-22, wherein the subject has the condition at the time of administering the steroid therapy or withholding the steroid therapy.

Embodiment 26. The method of any one of embodiments 1-22, wherein the subject does not have the condition at the time of administering the steroid therapy or withholding the steroid therapy.

Embodiment 27. A method for treating a hyper-proliferative skin condition of a subject, comprising administering radiation therapy to the subject subsequent to surgical excision (of some or all of the abnormal tissue).

Embodiment 28. The method of embodiment 27, wherein the radiation therapy is administered after the surgical wound(s) have healed.

Embodiment 29. The method of embodiment 27 or 28, wherein the radiation therapy is administered up to 3 months post-surgery.

Embodiment 30. The method of any one of embodiments 27 to 29, wherein the radiation therapy is administered to the subject in a single (unfractionated) dose.

Embodiment 31. The method of any one of embodiments 27 to 29, wherein the radiation therapy is administered in an effective amount to avoid recurrence of the hyper-proliferative skin condition at the anatomical site on the subject.

Embodiment 32. The method of any one of embodiments 27 to 31, wherein the radiation therapy is low (50 kV) to moderate (320 kV) energy.

Embodiment 33. The method of any one of embodiments 27 to 32, wherein the radiation therapy comprises X-ray energy. Embodiment 34. The method of any one of embodiments 27 to 33, wherein the radiation therapy comprises a single 8Gy dose of superficial low energy (50 kV) radiation.

Embodiment 35. The method of any one of embodiments 27 to 34, wherein the radiation therapy comprises a single 8Gy dose of superficial low energy (50 kV) X-ray radiation.

Embodiment 36. The method of any one of embodiments 27 to 35, wherein the hyper-proliferative skin condition is one or more keloids, one or more hypertrophic scars, or one or more tumors.

Embodiment 37. The method of any one of embodiments 27 to 36, wherein the method does not include administration of a steroid to the subject.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Bran G M, Goessler U R, Hormann K, Riedel F, Sadick H. Keloids: current concepts of pathogenesis (review). Int J Mol Med 2009;24:283-93.
2. Nakaoka H, Miyauchi S, Miki Y. Proliferating activity of dermal fibroblasts in keloids and hypertrophic scars. Acta Derm Venereol 1995;75:102-4.
3. Teofoli P, Barduagni S, Ribuffo M, Campanella A, De Pita O, Puddu P. Expression of Bcl-2, p53, c-jun and c-fos protooncogenes in keloids and hypertrophic scars. J Dermatol Sci 1999;22:31-7.
4. Bock O, Schmid-Ott G, Malewski P, Mrowietz U. Quality of life of patients with keloid and hypertrophic scarring. Arch Dermatol Res 2006;297:433-8.
5. Lee S S, Yosipovitch G, Chan Y H, Goh C L. Pruritus, pain, and small nerve fiber function in keloids: a controlled study. J Am Acad Dermatol 2004;51:1002-6.
6. Barrett J. Keloid. In: D. Bergsma editor. Birth defect compendium. Baltimore: Williams and Wilken Company; 1973. p. 553.
7. Berman B, Flores F. Recurrence rates of excised keloids treated with postoperative triamcinolone acetonide injections or interferon alfa-2b injections. J Am Acad Dermatol
8. Sclafani A P, Gordon L, Chadha M, Romo T, 3rd. Prevention of earlobe keloid recurrence with postoperative corticosteroid injections versus radiation therapy: a randomized, prospective study and review of the literature. Dermatol Surg 1996;22:569-74.
9. Shaffer J J, Taylor S C, Cook-Bolden F. Keloidal scars: a review with a critical look at therapeutic options. J Am Acad Dermatol 2002;46:S63-97.
10. Arno A I, Gauglitz G G, Barret J P, Jeschke M G. Up-to-date approach to manage keloids and hypertrophic scars: a useful guide. Burns 2014;40:1255-66.
11. Bijlard E, Steltenpool S, Niessen F B. Intralesional 5-fluorouracil in keloid treatment: a systematic review. Acta Derm Venereol 2015;95:778-82.
12. Berman B, Bieley HC. Adjunct therapies to surgical management of keloids. Dermatol Surg 1996;22:126-30.
13. Jung J Y, Roh M R, Kwon Y S, Chung K Y. Surgery and perioperative intralesional corticosteroid injection for treating earlobe keloids: a korean experience. Ann Dermatol 2009;21:221-5.
14. Shons A R, Press B H. The treatment of earlobe keloids by surgical excision and postoperative triamcinolone injection. Ann Plast Surg 1983;10:480-2.
15. Kiil J. Keloids treated with topical injections of triamcinolone acetonide (kenalog). Immediate and long-term results. Scand J Plast Reconstr Surg 1977;11:169-72.
16. Ledon J A, Savas J, Franca K, Chacon A, Nouri K. Intralesional treatment for keloids and hypertrophic scars: a review. Dermatol Surg 2013;39:1745-57.
17. Wilson A M. Eradication of keloids: Surgical excision followed by a single injection of intralesional 5-fluorouracil and botulinum toxin. Can J Plast Surg 2013;21:87-91.
18. Litrowski N, Boullie M C, Dehesdin D, De Barros A, Joly P. Treatment of earlobe keloids by surgical excision and cryosurgery. J Eur Acad Dermatol Venereol 2014;28:1324-31.
19. Emad M, Omidvari S, Dastgheib L, Mortazavi A, Ghaem H. Surgical excision and immediate postoperative radiotherapy versus cryotherapy and intralesional steroids in the management of keloids: a prospective clinical trial. Med Princ Pract 2010;19:402-5.
20. Flickinger J C. A radiobiological analysis of multicenter data for postoperative keloid radiotherapy. Int J Radiat Oncol Biol Phys 2011;79:1164-70.
21. Kim J, Lee S H. Therapeutic results and safety of postoperative radiotherapy for keloid after repeated Cesarean section in immediate postpartum period. Radiat Oncol J 2012;30:49-52.
22. Lee S Y, Park J. Postoperative electron beam radiotherapy for keloids: treatment outcome and factors associated with occurrence and recurrence. Ann Dermatol 2015;27:53-8.
23. Ragoowansi R, Cornes P G, Moss A L, Glees J P. Treatment of keloids by surgical excision and immediate postoperative single-fraction radiotherapy. Plast Reconstr Surg 2003;111:1853-9.
24. Ogawa R, Huang C, Akaishi S, Dohi T, Sugimoto A, Kuribayashi S et al. Analysis of surgical treatments for earlobe keloids: analysis of 174 lesions in 145 patients. Plast Reconstr Surg 2013;132:818e-25e.
25. Gauglitz GG. Management of keloids and hypertrophic scars: current and emerging options. Clin Cosmet Investig Dermatol 2013;6:103-14.
26. Mankowski P, Kanevsky J, Tomlinson J, Dyachenko A, Luc M. Optimizing Radiotherapy for Keloids: A Meta-Analysis Systematic Review Comparing Recurrence Rates Between Different Radiation Modalities. Ann Plast Surg 2017;78:403-11.
27. Lagares D, Busnadiego O, Garcia-Fernandez R A, Kapoor M, Liu S, Carter DE et al. Inhibition of focal adhesion kinase prevents experimental lung fibrosis and myofibroblast formation. Arthritis Rheum 2012;64:1653-64.
28. Rosell-Garcia T, Rodriguez-Pascual F. Enhancement of collagen deposition and cross-linking by coupling lysyl oxidase with bone morphogenetic protein-1 and its application in tissue engineering. Sci Rep 2018;8:10780.
29. Tirgan M. Worsening of keloids after intralesional injections. Journal of the American Academy of Dermatology 2013;68:AB68.
30. Tirgan M H. Intralesional Triamcinolone Acetonide in the Treatment of Keloid Lesions—Can the treatment be harmful to some patients? Results of an online survey. International Journal of Keloid Research 2017;1:21-8.
31. Sruthi K, Chelakkot P G, Madhavan R, Nair R R, Dinesh M. Single-fraction radiation: A promising adjuvant therapy to prevent keloid recurrence. J Cancer Res Ther 2018;14:1251-5.
32. Xu J, Yang E, Yu N Z, Long X. Radiation Therapy in Keloids Treatment: History, Strategy, Effectiveness, and Complication. Chin Med J (Engl) 2017;130:1715-21.
33. Song C, Wu H G, Chang H, Kim I H, Ha S W. Adjuvant single-fraction radiotherapy is safe and effective for intractable keloids. J Radiat Res 2014;55:912-6.
34. Ishihara H, Yoshimoto H, Fujioka M, Murakami R, Hirano A, Fujii T et al. Keloid fibroblasts resist ceramide-induced apoptosis by overexpression of insulin-like growth factor I receptor. J Invest Dermatol 2000;115:1065-71.
35. Daian T, Ohtsuru A, Rogounovitch T, Ishihara H, Hirano A, Akiyama-Uchida Y et al.

Insulin-like growth factor-I enhances transforming growth factor-beta-induced extracellular matrix protein production through the P38/activating transcription factor-2 signaling pathway in keloid fibroblasts. J Invest Dermatol 2003;120:956-62.
36. Phan T T, See P, Tran E, Nguyen T T, Chan S Y, Lee S T et al. Suppression of insulin-like growth factor signalling pathway and collagen expression in keloid-derived fibroblasts by quercetin: its therapeutic potential use in the treatment and/or prevention of keloids. Br J Dermatol 2003;148:544-52.
37. Dienus K, Bayat A, Gilmore BF, Seifert O. Increased expression of fibroblast activation protein-alpha in keloid fibroblasts: implications for development of a novel treatment option. Arch Dermatol Res 2010;302:725-31.
38. Luo S, Benathan M, Raffoul W, Panizzon R G, Egloff D V. Abnormal balance between proliferation and apoptotic cell death in fibroblasts derived from keloid lesions. Plast Reconstr Surg 2001;107:87-96.
39. Russell S B, Russell J D, Trupin K M, Gayden A E, Opalenik S R, Nanney L B et al. Epigenetically altered wound healing in keloid fibroblasts. J Invest Dermatol 2010;130:2489-96.
40. Zhang M Z, Dong X H, Guan E L, Si L B, Zhuge R Q, Zhao P X et al. A comparison of apoptosis levels in keloid tissue, physiological scars and normal skin. Am J Transl Res 2017;9:5548-57.
41. Tosa M, Ghazizadeh M, Shimizu H, Hirai T, Hyakusoku H, Kawanami 0. Global gene expression analysis of keloid fibroblasts in response to electron beam irradiation reveals the involvement of interleukin-6 pathway. J Invest Dermatol 2005;124:704-13.
42. Truong K, Bradley S, Baginski B, Wilson J R, Medlin D, Zheng L et al. The effect of well-characterized, very low-dose x-ray radiation on fibroblasts. PLoS One 2018;13 :e0190330.

We claim:

1. A method for determining whether or not a subject will respond to a steroid therapy, the method comprising: (a) administering a steroid to a sample of cells obtained from the subject in vitro; (b) determining the effect of the steroid on proliferation of the cells, wherein a decrease in proliferation caused by the steroid is indicative of responsiveness to steroid therapy, wherein no effect on cell proliferation is indicative of a lack of responsiveness to steroid therapy, and wherein an increase in proliferation caused by the steroid is indicative of an adverse effect associated with steroid therapy.

2. The method of claim 1, wherein the sample of cells comprise one or more cell types selected from among fibroblasts, epithelial cells, keratinocytes, and melanocytes.

3. The method of claim 1, wherein the subject has a condition, for which a steroid is a treatment, at the time the sample of cells is obtained from the subject.

4. The method of claim 1, wherein the subject does not have a condition, for which a steroid is a treatment option, at the time the sample of cells is obtained from the subject.

5. The method of claim 1, wherein cells of the cell sample comprise normal cells.

6. The method of claim 1, wherein the cells of the cell sample comprise abnormal or diseased cells.

7. The method of claim 1, wherein the sample of cells comprises cells of a keloid.

8. The method of claim 1, wherein the sample of cells comprises buccal cells, or cells of a surgical biopsy.

9. The method of claim 1, further comprising, prior to administering the steroid to the sample of cells, dissociating cells of the sample of cells.

10. The method of claim 1, further comprising, prior to administering the steroid to the sample of cells, culturing the cells under conditions sufficient to produce an outgrowth of primary cells.

11. The method of claim 10, wherein said administering of (a) comprises administering the steroid to the outgrown primary cells.

12. The method of claim 1, wherein said determining of (b) comprises conducting a cell proliferation assay.

13. The method of claim 1, wherein the cells are seeded onto a solid support prior to administering the steroid and determining the effect of the steroid on cell proliferation.

14. The method of claim 1, wherein the steroid comprises a corticosteroid.

15. The method of claim 3, wherein the condition is an immune disorder, inflammatory disorder, hyper-proliferative disorder, or dermatological disorder.

16. A method for selecting a therapy for a subject having a condition, comprising:
receiving results of an in vitro test for determining the effect of a steroid on proliferation of a sample of cells obtained from the subject, or carrying out the method of claim 1; and
selecting a steroid therapy for the subject if there is a decrease in cell proliferation caused by the steroid, or withholding steroid therapy from the subject and optionally selecting a non-steroid therapy for the subject if there is no effect on cell proliferation, or an increase in cell proliferation.

17. The method of claim 16, wherein the in vitro test comprises: (a) administering a steroid to a sample of cells obtained from the subject in vitro; (b) determining the effect of the steroid on proliferation of the cells, wherein a decrease in proliferation caused by the steroid is indicative of responsiveness to steroid therapy, wherein no effect on cell proliferation is indicative of a lack of responsiveness to steroid therapy, and wherein an increase in proliferation caused by the steroid is indicative of an adverse effect associated with steroid therapy.

18. The method of claim 16, wherein the condition is an immune disorder, inflammatory disorder, hyper-proliferative disorder, or dermatological disorder.

19. A method for treating a condition of a subject, comprising:
receiving results of an in vitro test for determining the effect of a steroid on proliferation of a sample of cells obtained from the subject, or carrying out the method of claim 1; and
administering a steroid therapy to the subject if there is a decrease in cell proliferation caused by the steroid, or withholding steroid therapy from the subject and optionally administering a non-steroid therapy to the subject if there is no effect on cell proliferation, or an increase in cell proliferation.

20. The method of claim 19, wherein the condition is an immune disorder, inflammatory disorder, hyper-proliferative disorder, or dermatological disorder.
